(19) United States
(12) Patent Application Publication
(10) Pub. No.: US 2026/0092315 A1
(43) Pub. Date: Apr. 2, 2026

(54) COMPOSITIONS AND METHODS FOR DISCOVERING GENE REGULATION

(71) Applicant: UNIVERSITY OF UTAH RESEARCH FOUNDATION, Salt Lake City, UT (US)

(72) Inventors: Alex BOTT, Salt Lake City, UT (US); Jared P. RUTTER, East Salt Lake City, UT (US)

(21) Appl. No.: 19/107,516

(22) PCT Filed: Dec. 13, 2023
(86) PCT No.: PCT/US2023/083852

§ 371 Date: Feb. 27, 2025

Related U.S. Application Data
(60) Provisional application No. 63/432,191, filed on Dec. 13, 2022.
(60) Provisional application No. 63/482,162, filed on Jan. 30, 2023.

(51) Int. Cl. C12Q 1/6841; C12Q 1/6874

(52) U.S. Cl. CPC C12Q 1/6841; C12Q 1/6874; C12Q 2600/158

(57) ABSTRACT

Disclosed are methods of identifying a regulatory factor that regulates the abundance of a target RNA comprising introducing to a population of cells a plurality of single guide RNAs (sgRNAs), wherein the sgRNAs are specific for one or more nucleic acid sequences capable of encoding the regulatory factor in the population of cells; performing fluorescent in situ hybridization (FISH) on the population cells, using a probe specific to a target RNA in the population of cells; identifying cells in the population of cells that have altered abundance of the target RNA; sequencing DNA from the population of cells that have altered abundance of the target RNA; and identifying, based on the sequencing, sgRNAs that are specific to a nucleic acid sequence capable of encoding the regulatory factor that regulates abundance of the target RNA; thereby identifying a regulatory factor that regulates abundance of the target RNA.

### CROSS-REFERENCE TO RELATED APPLICATIONS

This application claims the benefit of U.S. Provisional Patent Application No. 63/432,191, filed Dec. 13, 2022, and U.S. Provisional Patent Application No. 63/482,162, filed Jan. 30, 2023, each of which is incorporated by reference herein in its entirety.

### STATEMENT REGARDING FEDERALLY SPONSORED RESEARCH

This invention was made with government support under CA212445, and GM131854 awarded by the National Institutes of Health. The government has certain rights in the invention.

### BACKGROUND

A major goal in understanding gene regulation is identifying the set of factors that control each gene's expression. Many aspects of a gene's regulation can only be understood in its native genomic context; however, this places many limitations on the types of experiments that can be performed as well as how easily they can be applied to the study of any and all genes. To address this problem, we have developed Targeted Readout to Understand Transcription via Fluorescent In Situ Hybridization (TRoUT-FISH) as a straightforward and unbiased means to identify all factors that regulate a gene at the level of transcript abundance. We have deployed this technology—which combines fluorescent in situ hybridization, flow cytometry, and CRISPR screening—to elucidate mechanisms of basal gene expression, drug-induced transcription, and cellular responses to metabolic challenges—including the systemic dissection of glycolysis, an ancient metabolic pathway. We envision TRoUT-FISH as a platform to answer fundamental questions about gene regulation—some of which have historically been difficult to tackle—including disentangling redundant networks like the integrated stress response, illuminating cooperativity between transcriptional co-regulators, and annotating the regulome.

### BRIEF SUMMARY

Disclosed are methods of identifying a regulatory factor that regulates the abundance of a target RNA comprising introducing to a population of cells a plurality of single guide RNAs (sgRNAs), wherein the sgRNAs are specific for one or more nucleic acid sequences capable of encoding the regulatory factor in the population of cells; performing fluorescent in situ hybridization (FISH) on the population of cells, using a probe specific to a target RNA in the population of cells; identifying cells in the population of cells that have altered abundance of the target RNA; sequencing DNA from the population of cells that have altered abundance of the target RNA; and identifying, based on the sequencing, sgRNAs that are specific to a nucleic acid sequence capable of encoding the regulatory factor that regulates abundance of the target RNA; thereby identifying a regulatory factor that regulates abundance of the target RNA.

Disclosed are methods of identifying a regulatory factor that regulates abundance of a target RNA effected by a candidate compound comprising contacting a first population of cells with a candidate compound; identifying a target RNA that is modulated by the candidate compound; preparing a probe specific to the target RNA modulated by the candidate compound; introducing to a second population of cells a plurality of single guide RNAs (sgRNAs), wherein the sgRNAs are specific for one or more target genes in the second population of cells; performing fluorescent in situ hybridization (FISH) on the second population of cells, using the probe specific to the target RNA; identifying cells in the second population of cells that have altered expression of the target RNA; sequencing DNA from the cells of step f) using primers specific to the plurality of sgRNAs; identifying, based on the sequencing, sgRNAs that are specific to a nucleic acid sequence capable of encoding the regulatory factor that regulates the abundance of the target RNA, and thereby identifying a regulatory factor that regulates expression of the target RNA effected by the candidate compound.

Disclosed are methods of identifying a candidate compound that regulates abundance of a target RNA comprising optionally introducing a barcode to a population of cells; contacting the population of cells with a candidate compound; performing fluorescent in situ hybridization (FISH) on the population of cells contacted with the candidate compound, using a probe specific to a target RNA in the population of cells; identifying cells in the population of cells that have altered abundance of the target RNA; sequencing DNA from the cells of identified as having an abundance of the target RNA using primers specific to the barcode; identifying, based on sequencing, the barcode in each cell, wherein the barcode corresponds to the candidate compound contacted to the population of cells having that barcode; thereby identifying a candidate compound that regulates the abundance of the target RNA.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIGS. 1A, 1B, and 1C show gene regulation complexity. FIG. 1A) Schematic of regulatory nodes controlling gene expression. FIG. 1B) Relationship between a modulator and elucidator. FIG. 1C) Relationship between a signal, a sensor, and elucidator. Interaction from B can be contained in the multi-arrow.

FIG. 2 shows Targeted Readout to Understand Transcription via Fluorescent In Situ Hybridization (TRoUT-FISH).

FIGS. 3A-3D show TRoUT-FISH positively identifies known mechanisms of gene regulation. Treatment with Nutlin-3a FIG. 3A) stabilizes p53 protein and FIG. 3B) induces CDKN1A transcription. FIG. 3C) FISH and flow cytometry detects drug induced changes in CDKN1A mRNA levels. FIG. 3D) MAGeCK analysis of Nutlin-3a TRoUT-FISH with CDKN1A as the elucidator. ****p<0.0001 T-test.

FIGS. 4A-4C show TRoUT-FISH identifies MYC and BRD4 as regulators of glycolysis. FIG. 4A) TRoUT-FISH data from ENO1 and PGK1 screens. B,C) K562 cells were treated with 1 uM JQ1 for 6 h. FIG. 4B) MYC protein level measured by immunoblot and FIG. 4C) transcripts measured by qPCR. **p<0.01, ****p<0.0001 T-test.

FIGS. 5A-5C show TRoUT-FISH identifies lineage specific transcription factors. FIG. 5A) ENO1 and GPI screens in Mewo skin cell line. FIG. 5B) ENO1 and PGK1 screens in K562 cells. FIG. 5C) Genome browser track of ATAC-seq reads from Mewo and K562 cells near ENO1. Aggregate ChIP-seq peaks for MITF or KLF1 across samples in the ReMap database.

FIGS. 6A-6B show TRoUT-FISH identifies lineage specific transcription factors. FIG. 6A) Upset plot comparing significant hits (FDR<0.05) from K562 PGK1 and ENO1 TRoUT-FISH screens. FIG. 6B) Upset plot comparing significant hits (FDR<0.05) from K562 ENO1 and Mewo ENO1 TRoUT-FISH screens. FIG.

FIGS. 7A and 7B show SLC16A6 is upregulated upon nutrient deprivation and enriched in skin. FIG. 7A) Deprivation of branched chain amino acids induces SLC16A6. ***p<0.001 ANVOA with Dunnett's multiple comparison test. FIG. 7B) SLC16A6 expression grouped by tissue. Number of cell lines by tissue indicated.

FIGS. 8A-8C show SLC16A6 is regulated by MITF. FIG. 8A) MAGeCK analysis of SLC16A6 TRoUT-FISH experiment identifies SOX10 and MITF. FIG. 8B) CRISPR deletion of MITF reduces SLC16A6 expression. FIG. 8C) MITF overexpression induces SLC16A6 expression. ****p<0.0001 Wald test with BH adjustment.

FIG. 9 shows TFE3 and TFEB are sufficient to induce SLC16A6 expression. ***p<0.001 ANVOA with Dunnett's multiple comparison test.

FIGS. 10A-10F show TRoUT-FISH identifies BRDs as regulators of ERRFI1. FIG. 10A) Methionine deprivation induces ERRFI1. ****p<0.0001 ANVOA with Dunnett's multiple comparison test. FIG. 10B) MAGeCK analysis of ERRFI1 TRoUT-FISH identifies BRD2. FIG. 10C) JQ1 decreases ERRFI1 expression. ****p<0.0001 Wald test with BH adjustment. FIG. 10D) Genome browser of H3K27ac and BRD4 enrichment upstream of ERRFI1. FIG. 10E) Schematic of comparisons for TRoUT-FISH screen with multiple conditions. FIG. 10F) MAGeCK analysis comparing low tails (magenta and cyan in E) of ERRFI1 TRoUT-FISH.

FIG. 11A-C show Method to Associate Compounds with Altered RNA levels via Fluorescent In Situ Hybridization (MACAREL-FISH). FIG. 11A) Schematic of MACAREL-FISH. FIG. 11B) Schematic of sorting strategy. Magenta boxes indicate the sorted population. Low and high populations were sorted from the Mix 50:50 population. FIG. 11C) MACAREL-FISH recovery of the indicated barcodes from each population, identifying Nutlin-3a as an activator of CDKN1A RNA abundance.

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a peptide is disclosed and discussed and a number of modifications that can be made to a number of molecules including the amino acids are discussed, each and every combination and permutation of the peptide and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a sgRNA” includes a plurality of such sgRNAs, reference to “the primer” is a reference to one or more primers and equivalents thereof known to those skilled in the art, and so forth.

The word “or” as used herein means any one member of a particular list and also includes any combination of members of that list.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### B. Methods

Disclosed are methods of identifying the players in RNA regulation. In some aspects, if a target RNA is known, the disclosed methods can identifying what regulatory factors play a role upstream in altering the abundance of the target RNA. In some aspects, if a compound is known, the disclosed methods can identify what target RNA is modulated by the compound and then can identifying what regulatory factors play a role in the compound altering the abundance of the target RNA. In some aspects, the disclosed methods can identify a candidate compound as having a role in altering the abundance of a target RNA.

### 1. Identifying Regulatory Factors That Regulate a Target RNA

Disclosed are methods of identifying a regulatory factor that regulates the abundance of a target RNA comprising introducing to a population of cells a plurality of single guide RNAs (sgRNAs), wherein the sgRNAs are specific for one or more nucleic acid sequences capable of encoding the regulatory factor in the population of cells; performing fluorescent in situ hybridization (FISH) on the population of cells, using a probe specific to a target RNA in the population of cells; identifying cells in the population of cells that have altered abundance of the target RNA; sequencing DNA from the population of cells that have altered abundance of the target RNA; and identifying, based on the sequencing, sgRNAs that are specific to a nucleic acid sequence capable of encoding the regulatory factor that regulates abundance of the target RNA; thereby identifying a regulatory factor that regulates abundance of the target RNA.

In some aspects, the population of cells used in the disclosed methods can be mammalian cells. In some aspects, the population of cells can be human cells or mouse cells. In some aspects, the population of cells can be healthy cells or disease cells. In some aspects, disease cells can be, but are not limited to, cancer cells, genetic disease cells, and metabolic disease cells. In some aspects, healthy cells can be any cells with no known disease or condition. In some aspects, disease cells can be any cells with a known disease or condition. In some aspects, cancer cells can be any cancer cell, such as, but not limited to, breast cancer cells, lung cancer cells, glioma cells, pancreatic cancer cells, liver cancer cells, or colon cancer cells. In some aspects, disease cells can be any metabolic disease/condition cell, for example, a metabolic disease/condition can be, but is not limited to, inborn errors of metabolism. In some aspects, genetic disease cells can be cells from any genetic disease, such as, Huntingtons, cystic fibrosis, or sickle cell. In some aspects, the cells are primary cells. In some aspects, the cells are a cell line.

In some aspects, previous methods were known to target non-coding regions of the genome to gain positional information about regulation. For example, earlier studies can help understand what non-coding genomic regions are important regulatory regions. In some aspects, these earlier methods use gRNA libraries that tile across non-coding areas of the genome proximal to their target of interest. In some aspects, this approach can be useful to understand positional regulatory information. For example, earlier studies targeted non-coding DNA proximal to their gene/RNA of interest. This is also known as cis-acting factors (enhancers, silencers, etc).

The disclosed methods address what molecular players (e.g. regulatory factors including trans-acting factors) are involved in regulating a gene by looking at target RNA transcribed from the gene. In some aspects, the disclosed methods use a sgRNA library that targets protein coding genes and other coding genes (i.e. long non-coding RNAs [IncRNAs]) that do not encode a protein but still produce a molecule that has a function and from this it can be determined what proteins, molecules, or genes are involved in regulating other target RNAs.

The disclosed methods can interrogate trans-acting factors (transcription factors, IncRNAs) as well as proteins that do not act on DNA directly. For example, the disclosed methods (sometimes referred to as TRoUT-FISH) can determine a regulatory axis consisting of a kinase that phosphorylates an E3 ubiquitin ligase that degrades a transcription factor. This cannot be performed using previously known techniques.

### i. Introducing sgRNAs

In some aspects, introducing to a population of cells a plurality of sgRNAs comprises introducing a sgRNA library. In some aspects, a sgRNA library has known known targets for each sgRNA. For example, a sgRNA library can target kinases or epigenetic factors, such as, but not limited to, chromatin modifiers, transcription factors, and transcriptional activators. In some aspects, using an epigenetic specific library can detect regulation at the level of chromatin modification, multi-protein transcriptional complexes, and direct DNA binding transcription factors.

In some aspects, introducing to a population of cells a plurality of sgRNAs comprises introducing to the population of cells a virus library, wherein each virus comprises a sgRNA. In some aspects, the viral library can be made from, but are not limited to, lentiviruses, retroviruses, adenoviruses, or adeno-associated viruses. In some aspects, introducing to a population of cells a plurality of sgRNAs comprises introducing to the population of cells a population of DNA constructs, wherein each DNA construct comprises a sgRNA. In some aspects, the DNA construct can be a plasmid. In some aspects, the plurality of sgRNAs can be introduced using viruses or DNA constructs.

In some aspects, introducing the plurality sgRNAs can comprising introducing a CRISPR system to the cells wherein the CRISPR system comprises a plurality of sgRNAs and a Cas protein, wherein a sgRNA and Cas protein complex at the sgRNA target. Thus, in some aspects, the population of cells comprise a Cas protein. For example, a cell line designed to express Cas can be used. In some aspects, the method can further comprise contacting the population of cells with a Cas protein or a gene capable of expressing a Cas protein prior to performing FISH. In some aspects, introducing sgRNAs to the population of cells can occur prior to, after, or simultaneously with a Cas protein or a gene capable of expressing a Cas protein. In some aspects, the Cas protein can be, but is not limited to, Cas9, Cas12a, and Cas13.

In some aspects, the CRISPR system uses a CRISPR nuclease to cut DNA, CRISPRi to interfere with gene expression, or CRISPRa to activate gene expression. Depending on which CRISPR system is used, in combination with what the regulatory factor does, can result in an increase or decrease of the abundance of target RNA.

### a. Crispr

In some aspects, the disclosed methods can use a CRISPR system wherein a Cas protein cleaves at least a portion of a nucleic acid sequence capable of encoding the regulatory factor after the sgRNA guides the Cas protein to the nucleic acid sequence. Thus, the cells then do not have a functional regulatory factor that would have been encoded by the nucleic acid sequence that was cleaved. In some aspects, the disclosed method then tests the cells without the functional regulatory factor to determine what effects it has on a target RNA.

### b. CRISPRi

In some aspects, the disclosed methods can use a CRSIPR interference (CRISPRi) system. CRISPRi uses a catalytically dead Cas (dCas) protein that lacks endonuclease activity fused to transcriptional repression machinery to regulate genes in an RNA-guided (e.g. sgRNA) manner. In some aspects, CRISPRi can repress transcription by blocking either transcriptional initiation or elongation. Thus, in some aspects, CRISPRi can be used in the disclosed methods by the sgRNA guiding a dCas to a site adjacent to the transcriptional start site, within the promoter, or exonic sequence of the nucleic acid sequence capable of encoding a regulatory factor. In some aspects, this can result in the regulatory factor not being transcribed and thus, no protein can be encoded which allows the disclosed method to test the cells without the functional regulatory factor to determine what effects it has on a target RNA.

### c. CRISPRa

In some aspects, the disclosed methods can use a CRSIPR activation (CRISPRa) system. Different from the CRISPR and CRISPRi described above, CRISPRa uses sgRNAs and a dCas fused to transcriptional activation machinery to activate a regulatory factor instead of repress or remove the regulatory factor. In some aspects, this can result in the regulatory factor being activated and thus, abundance of the encoded protein can be increased, which allows the disclosed method to test the cells determine what effects the presence of the regulatory factor has on a target RNA.

### ii. Performing FISH

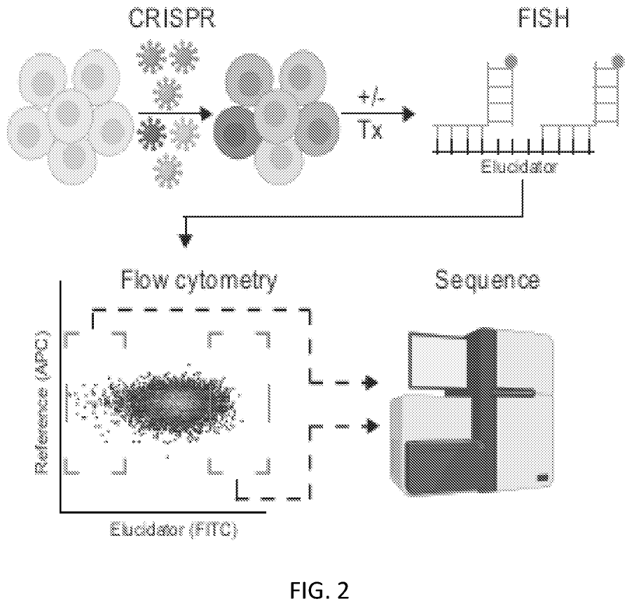

In some aspects, performing fluorescent in situ hybridization (FISH) on the population of cells, using a probe specific to a target RNA in the population of cells can comprise any known method of labeling the target RNA. In some aspects, the probe specific to a target RNA is directly labeled. For example, using a labeled probe that is specific to the target RNA can be performed by using a prelabeled probe and then contacting it with cells having the target RNA. In some aspects, a labeled probe that is specific to the probe specific to the target RNA can be used. For example, this can be referred to as sequential labeling wherein an unlabeled probe is specific to the target and a labeled probe then hybridizes to the unlabeled probe (see FIG. 2). In some aspects, hybridization chain reaction can be used to label. In some aspects, the labeled probe, whether it be a prelabeled probe that binds the target RNA or a labeled probe that binds another probe, can be fluorescently labeled. For example, the fluorescent label can be, but is not limited to, a fluorophore (e.g. red fluorescent protein, green fluorescent protein), biotin, or enzyme.

### iii. Identifying Cells With Altered Abundance of Target RNA

In some aspects, identifying cells in the population of cells that have altered abundance of the target RNA can comprise performing flow cytometry or fluorescence activated cell sorting (FACS). In some aspects, FACS allows for identifying and sorting cells based on the level of fluorescence which corresponds to the level of target RNA present in the cells.

In some aspects, cells can be sorted based on highest signal and lowest signal using Flow, wherein the lowest signal cells means that sgRNA knocked out something that is needed to increase expression of target RNA or for CRISPRa the system activated something that decreases expression of the target RNA. In some aspects, the highest signal means that sgRNA knocked out something that inhibits expression of target RNA and without it the target RNA is increased in abundance, however, for CRISPRa the system activated something that increases expression of the target RNA. In some aspects, DNA can be extracted from the sorted cells (high abundance target RNA and low abundance target RNA) and sequenced for sgRNA

In some aspects, cells having altered abundance of the target RNA can be identified using any known methods for identifying cells based on the presence or absence of a label. For example, in some aspects, northern blotting, quantitative real time PCR or RNA sequencing can be used.

### iv. Sequencing

In some aspects, sequencing DNA from the population of cells that have altered abundance of the target RNA comprises sequencing the sgRNAs. In some aspects, sequencing comprises contacting the DNA from the population of cells that have altered abundance of the target RNA with primers specific to a constant region upstream and downstream of the sgRNA on the plasmid. In some aspects, sequencing DNA from the population of cells that have altered abundance of the target RNA results in identification of the sgRNA introduced into that cell.

### v. Identifying sgRNA Equals Identifying Regulatory Factor Being Targeted by sgRNA

In some aspects, the identification, based on sequencing, of the sgRNA introduced into each cell can identify the regulatory factor that regulates abundance of the target RNA because the sgRNA is specific to a nucleic acid sequence capable of encoding the regulatory factor. Thus, in some aspects, there is a direct correlation between the sgRNA and the regulatory factor. In some aspects, the sgRNA is specific to a nucleic acid sequence capable of encoding a regulatory factor, therefore identifying the sgRNA present in a cell also identifies what regulatory factor (that was targeted by the sgRNA) affects the target RNA.

In some aspects, the regulatory factor can be any molecule that can alter the abundance of a target RNA. In some aspects, the regulatory factor can be, but is not limited to, a transcription factor, kinase, G protein, splicing machinery, chromatin modifier, phosphatase, enzyme, metabolic gene, metabolite transporter, chaperone, scaffolding protein, ligase, signaling machinery, degradation machinery.

### 2. Identifying a Target RNA Affected by a Candidate Compound

Disclosed are methods similar to the methods of identifying regulatory factors that regulate a target RNA except that first a known compound is used to determine what target RNA is altered and then once the target RNA is known then the methods of identifying regulatory factors that regulate the target RNA can be performed.

Disclosed are methods of identifying a regulatory factor that regulates abundance of a target RNA effected by a candidate compound comprising contacting a first population of cells with a candidate compound; identifying a target RNA that is modulated by the candidate compound; preparing a probe specific to the target RNA modulated by the candidate compound; introducing to a second population of cells a plurality of single guide RNAs (sgRNAs), wherein the sgRNAs are specific for one or more target genes in the second population of cells; performing fluorescent in situ hybridization (FISH) on the second population of cells, using the probe specific to the target RNA; identifying cells in the second population of cells that have altered expression of the target RNA; sequencing DNA from the cells of step f) using primers specific to the plurality of sgRNAs; identifying, based on the sequencing, sgRNAs that are specific to a nucleic acid sequence capable of encoding the regulatory factor that regulates the abundance of the target RNA, and thereby identifying a regulatory factor that regulates expression of the target RNA effected by the candidate compound.

In some aspects, the population of cells used in the disclosed methods can be mammalian cells. In some aspects, the population of cells can be human cells or mouse cells. In some aspects, the population of cells can be healthy cells or disease cells. In some aspects, disease cells can be, but are not limited to, cancer cells, genetic disease cells, and metabolic disease cells. In some aspects, healthy cells can be any cells with no known disease or condition. In some aspects, disease cells can be any cells with a known disease or condition. In some aspects, cancer cells can be any cancer cell, such as, but not limited to, breast cancer cells, lung cancer cells, glioma cells, pancreatic cancer cells, liver cancer cells, or colon cancer cells. In some aspects, disease relevant cells can be any metabolic disease/condition cell, for example, a metabolic disease/condition can be, but is not limited to, inborn errors of metabolism. In some aspects, genetic disease cells can be cells from any genetic disease, such as, Huntingtons, cystic fibrosis, or sickle cell.

In some aspects, the cells are primary cells. In some aspects, the cells are a cell line. In some aspects, the first population of cells and second population of cells are the same cell type.

### i. Contacting Cells With Candidate Compound

In some aspects, the methods comprise contacting a first population of cells with a candidate compound. In some aspects, the candidate compound can be a known compound but the one or more RNAs altered by the candidate compound are unknown. In some aspects, the candidate compound can be unknown.

In some aspects, contacting a first population of cells with a candidate compound comprises adding a candidate compound to the culture media of the first population of cells in culture. In some aspects, contacting refers to incubating a candidate compound with a population of cells. Thus, in some aspects, incubating can be for minutes, hours, or days. In some aspects, the candidate compound can be diluted into several dilutions for testing.

### ii. Identifying Target RNA Modulated by the Candidate Compound

In some aspects, the disclosed methods comprise identifying a target RNA that is modulated by the candidate compound. In some aspects, identifying a target RNA that is modulated by the candidate compound comprises performing RNA sequencing. In some aspects, after contacting the cells with a candidate compound, RNA can be extracted from the cells. In some aspects, RNA sequencing can be performed on the extracted RNA using known RNA sequencing techniques.

In some aspects, a candidate compound can increase or decrease RNAs in the cell and those RNAs can be used as the target RNA in the steps that follow.

### iii. Preparing a Probe

In some aspects, the methods comprise preparing a probe specific to the target RNA modulated by the candidate compound. In some aspects, once the target RNAs are identified (as those altered by the candidate compound) a probe can be prepared that specifically binds to the target RNA. In some aspects, there are known probes to the target RNA. In some aspects, the probe can be prepared based on the sequence of the target RNA.

### iv. Introducing sgRNAs

In some aspects, introducing to a population of cells a plurality of sgRNAs comprises introducing a sgRNA library. In some aspects, a sgRNA library has known known targets for each sgRNA. For example, a sgRNA library can target kinases or epigenetic factors, such as, but not limited to, chromatin modifiers, transcription factors, and transcriptional activators. In some aspects, using an epigenetic specific library can detect regulation at the level of chromatin modification, multi-protein transcriptional complexes, and direct DNA binding transcription factors.

In some aspects, introducing to a population of cells a plurality of sgRNAs comprises introducing to the population of cells a virus library, wherein each virus comprises a sgRNA. In some aspects, the viral library can be made from, but are not limited to, lentiviruses, retroviruses, adenoviruses, or adeno-associated viruses. In some aspects, introducing to a population of cells a plurality of sgRNAs comprises introducing to the population of cells a population of DNA constructs, wherein each DNA construct comprises a sgRNA. In some aspects, the DNA construct can be a plasmid. In some aspects, the plurality of sgRNAs can be introduced using viruses or DNA constructs.

In some aspects, introducing the plurality sgRNAs can comprising introducing a CRISPR system to the cells wherein the CRISPR system comprises a plurality of sgRNAs and a Cas protein, wherein a sgRNA and Cas protein complex at the sgRNA target. Thus, in some aspects, the population of cells comprise a Cas protein. For example, a cell line designed to express Cas can be used. In some aspects, the method can further comprise contacting the population of cells with a Cas protein or a gene capable of expressing a Cas protein prior to performing FISH. In some aspects, introducing sgRNAs to the population of cells can occur prior to, after, or simultaneously with a Cas protein or a gene capable of expressing a Cas protein. In some aspects, the Cas protein can be, but is not limited to, Cas9, Cas12a, and Cas13.

In some aspects, the CRISPR system uses a CRISPR nuclease to cut DNA, CRISPRi to interfere with gene expression, or CRISPRa to activate gene expression. Depending on which CRISPR system is used, in combination with what the regulatory factor does, can result in an increase or decrease of the abundance of target RNA.

### a. CRISPR

In some aspects, the disclosed methods can use a CRSIPR system wherein a Cas protein cleaves at least a portion of a nucleic acid sequence capable of encoding the regulatory factor after the sgRNA guides the Cas protein to the nucleic acid sequence. Thus, the cells then do not have a functional regulatory factor that would have been encoded by the nucleic acid sequence that was cleaved. In some aspects, the disclosed method then tests the cells without the functional regulatory factor to determine what effects it has on a target RNA.

### b. CRISPRi

In some aspects, the disclosed methods can use a CRSIPR interference (CRISPRi) system. CRISPRi uses a catalytically dead Cas (dCas) protein that lacks endonuclease activity fused to transcriptional repression machinery to regulate genes in an RNA-guided (e.g. sgRNA) manner. In some aspects, CRISPRi can repress transcription by blocking either transcriptional initiation or elongation. Thus, in some aspects, CRISPRi can be used in the disclosed methods by the sgRNA guiding a dCas to site within the promoter or exonic sequence of the nucleic acid sequence capable of encoding a regulatory factor. In some aspects, this can result in the regulatory factor not being transcribed and thus, no protein can be encoded which allows the disclosed method to test the cells without the functional regulatory factor to determine what effects it has on a target RNA.

### c. CRISPRa

In some aspects, the disclosed methods can use a CRSIPR activation (CRISPRa) system. Different from the CRISPR and CRISPRi described above, CRISPRa uses sgRNAs and a dCas9 fused to transcriptional activation machinery to activate a regulatory factor instead of repress or remove the regulatory factor. In some aspects, this can result in the regulatory factor being activated and thus, abundance of the encoded protein can be increased which allows the disclosed method to test the cells determine what effects the presence of the regulatory factor has on a target RNA.

### v. Performing FISH

In some aspects, performing fluorescent in situ hybridization (FISH) on the population of cells, using a probe specific to a target RNA in the population of cells can comprise any known method of labeling the target RNA. In some aspects, the probe specific to a target RNA is directly labeled. For example, using a labeled probe that is specific to the target RNA can be performed by using a prelabeled probe and then contacting it with cells having the target RNA. In some aspects, a labeled probe that is specific to the probe specific to the target RNA can be used. For example, this can be referred to as sequential labeling wherein an unlabeled probe is specific to the target and a labeled probe then hybridizes to the unlabeled probe (see FIG. 2). In some aspects, hybridization chain reaction can be used to label. In some aspects, the labeled probe, whether it be a prelabeled probe that binds the target RNA or a labeled probe that binds another probe, can be fluorescently labeled. For example, the fluorescent label can be, but is not limited to, a fluorophore (e.g. red fluorescent protein, green fluorescent protein), biotin, or enzyme.

### vi. Identifying Cells With Altered Abundance of Target RNA

In some aspects, the disclosed methods comprise identifying cells in the second population of cells that have altered abundance of the target RNA. In some aspects, identifying cells in the second population of cells that have altered abundance of the target RNA can comprise performing flow cytometry or fluorescence activated cell sorting (FACS). In some aspects, FACS allows for identifying and sorting cells based on the level of fluorescence which corresponds to the level of target RNA present in the cells.

In some aspects, cells can be sorted based on highest signal and lowest signal using Flow, wherein the lowest signal cells means that sgRNA knocked out something that is needed to increase expression of target RNA or for CRISPRa the system activated something that decreases expression of the target RNA. In some aspects, the highest signal means that sgRNA knocked out something that inhibits expression of target RNA and without it the target RNA is increased in abundance, however, for CRISPRa the system activated something that increases expression of the target RNA. In some aspects, DNA can be extracted from the sorted cells (high abundance target RNA and low abundance target RNA) and sequenced for sgRNA.

In some aspects, cells having altered abundance of the target RNA can be identified using any known methods for identifying cells based on the presence or absence of a label. For example, in some aspects, northern blotting, quantitative real time PCR or RNA sequencing can be used.

### vii. Sequencing

In some aspects, sequencing DNA from the population of cells that have altered abundance of the target RNA comprises sequencing the sgRNAs. In some aspects, sequencing comprises contacting the DNA from the population of cells that have altered abundance of the target RNA with primers specific to a constant region upstream and downstream of the sgRNA on the plasmid. In some aspects, sequencing DNA from the population of cells that have altered abundance of the target RNA results in identification of the sgRNA introduced into that cell.

### viii. Identifying sgRNA Equals Identifying Regulatory Factor Being Targeted by sgRNA

In some aspects, the identification, based on sequencing, of the sgRNA introduced into each cell can identify the regulatory factor that regulates abundance of the target RNA because the sgRNA is specific to a nucleic acid sequence capable of encoding the regulatory factor. Thus, in some aspects, there is a direct correlation between the sgRNA and the regulatory factor. In some aspects, the sgRNA is specific to a nucleic acid sequence capable of encoding a regulatory factor, therefore identifying the sgRNA present in a cell also identifies what regulatory factor (that was targeted by the sgRNA) affects the target RNA.

In some aspects, the regulatory factor can be any molecule that can alter the abundance of a target RNA. In some aspects, the regulatory factor can be, but is not limited to, a transcription factor, kinase, G protein, splicing machinery, chromatin modifier, phosphatase, enzyme, metabolic gene, metabolite transporter, chaperone, scaffolding protein, ligase, signaling machinery.

### 3. Identifying a Candidate Compound That Regulates a Target RNA

Disclosed are methods of identifying a candidate compound that regulates abundance of a target RNA comprising optionally introducing a barcode to a population of cells; contacting the population of cells with a candidate compound; performing fluorescent in situ hybridization (FISH) on the population of cells contacted with the candidate compound, using a probe specific to a target RNA in the population of cells; identifying cells in the population of cells that have altered abundance of the target RNA; sequencing DNA from the cells of identified as having an abundance of the target RNA using primers specific to the barcode; identifying, based on sequencing, the barcode in each cell, wherein the barcode corresponds to the candidate compound contacted to the population of cells having that barcode; thereby identifying a candidate compound that regulates the abundance of the target RNA.

In some aspects, the population of cells used in the disclosed methods can be mammalian cells. In some aspects, the population of cells can be human cells or mouse cells. In some aspects, the population of cells can be healthy cells or disease cells. In some aspects, disease cells can be, but are not limited to, cancer cells, genetic disease cells, and metabolic disease cells. In some aspects, healthy cells can be any cells with no known disease or condition. In some aspects, disease cells can be any cells with a known disease or condition. In some aspects, cancer cells can be any cancer cell, such as, but not limited to, breast cancer cells, lung cancer cells, glioma cells, pancreatic cancer cells, liver cancer cells, or colon cancer cells. In some aspects, disease cells can be any metabolic disease/condition cell, for example, a metabolic disease/condition can be, but is not limited to, inborn errors of metabolism. In some aspects, genetic disease cells can be cells from any genetic disease, such as, Huntingtons, cystic fibrosis, or sickle cell.

In some aspects, the cells are primary cells. In some aspects, the cells are a cell line.

### i. Introducing a Barcode

In some aspects, introducing a barcode to a population cells comprises administering a plurality of DNA constructs to the population of cells, wherein each DNA construct comprises a unique barcode. In some aspects, the plurality of DNA constructs are linear or circular. In some aspects, the plurality of DNA constructs is a plurality of plasmids.

In some aspects, after introducing a plurality of DNA constructs to the population of cells, the cells can be fixed. In some aspects, the plurality of DNA constructs does not enter the cells. In some aspects, the plurality of DNA constructs contacts the external surface of the cells. In some aspects, if the cells are fixed, the barcoding is performed after contacting the cells with a candidate compound.

In some aspects, barcoding can be performed using any known method. For example, in some aspects a barcode can be on an antibody and then the antibody (specific to the cell) is contacted with a cell. In some aspects, viral vectors can contain unique barcodes so that when they infect a cell the barcode is inserted/integrated into the cellular DNA. In some aspects, barcoding can comprise recombinases in living cells or molecular typewriters using Cas proteins to make a series of mutants.

In some aspects, introducing a barcode to a population cells can be performed before, after, or simultaneously with the step of contacting the population of cells with a candidate compound.

In some aspects, a barcode is a unique nucleic acid sequence allowing a different barcode to be contacted to different cells. In some aspects, a barcode can be, but is not limited to, a nucleic acid sequence have at least 2, 3, 4, 5, 6, 7, 8, 9, 10, 11, 12, 13, 14, 15, 16, 17, 18, 19, or 20 base pairs. In some aspects, a barcode can comprise 20, 25, 30, 35, 40 or more base pairs.

In some aspects, introducing a barcode to a population cells can be an optional step. In some aspects, a cell line can be used that already has barcodes. Thus, in some aspects, the first step of the disclosed methods would be contacting the population of cells having barcodes with a candidate compound.

In some aspects, the barcode is nucleic acid based or protein based.

### ii. Contacting Cells with Candidate Compound

In some aspects, the disclosed methods comprise contacting the population of cells with a candidate compound. In some aspects, contacting refers to adding a candidate compound to the culture media of one or more cells in culture. In some aspects, contacting refers to incubating a candidate compound with a population of cells. Thus, in some aspects, incubating can be for minutes, hours, or days. In some aspects, the candidate compound can be diluted into several dilutions for testing.

In some aspects, a candidate compound be, but is not limited to, a drug, fragment molecule, metabolite, or natural compound. For example, in some aspects, a candidate compound can be a cell type different from the cell being contacted with the candidate compound (e.g. other cell type).

### iii. Performing FISH

In some aspects, performing fluorescent in situ hybridization (FISH) on the population of cells, using a probe specific to a target RNA in the population of cells can comprise any known method of labeling the target RNA. In some aspects, the probe specific to a target RNA is directly labeled. For example, using a labeled probe that is specific to the target RNA can be performed by using a prelabeled probe and then contacting it with cells having the target RNA. In some aspects, a labeled probe that is specific to the probe specific to the target RNA can be used. Thus, in some aspects, the methods can further comprise, after contacting cells with a probe specific to a target RNA, contacting the population of cells with a labeled probe specific to the probe specific to the target RNA. For example, this can be referred to as sequential labeling wherein an unlabeled probe is specific to the target and a labeled probe then hybridizes to the unlabeled probe (see FIG. 2). In some aspects, hybridization chain reaction can be used to label. In some aspects, the labeled probe, whether it be a prelabeled probe that binds the target RNA or a labeled probe that binds another probe, can be fluorescently labeled. For example, the fluorescent label can be, but is not limited to, a fluorophore (e.g. red fluorescent protein, green fluorescent protein), biotin, or enzyme.

### iv. Identifying Cells With Altered Abundance of Target RNA

In some aspects, the methods comprise identifying cells in the population of cells that have altered abundance of the target RNA. In some aspects, identifying cells in the population of cells that have altered abundance of the target RNA can comprise performing flow cytometry or fluorescence activated cell sorting (FACS). In some aspects, FACS allows for identifying and sorting cells based on the level of fluorescence which corresponds to the level of target RNA present in the cells.

In some aspects, cells can be sorted based on highest signal and lowest signal using Flow, wherein the lowest signal cells means that the candidate compound decreased something that is needed to increase expression of target RNA or increased something that decreases expression of the target RNA. In some aspects, the highest signal means that the candidate compound decreased something that inhibits expression of target RNA and without it the target RNA is in abundance, or the candidate compound increased something that increases expression of the target RNA. In some aspects, DNA can be extracted from the sorted cells (high abundance target RNA and low abundance target RNA) and sequenced for barcodes.

In some aspects, cells having altered abundance of the target RNA can be identified using any known methods for identifying cells based on the presence or absence of a label. For example, in some aspects, northern blotting, quantitative real time PCR or RNA sequencing can be used.

### v. Sequencing

In some aspects, sequencing DNA from the population of cells that have altered abundance of the target RNA comprises sequencing the barcode. In some aspects, sequencing comprises contacting the DNA from the population of cells that have altered abundance of the target RNA with primers specific to a constant region upstream and downstream of the barcode. In some aspects, sequencing DNA from the population of cells that have altered abundance of the target RNA results in identification of the barcode introduced into that cell.

In some aspects, the barcode is present on a nucleic acid sequence, therefore the regions upstream and downstream of the barcode are constant regions on the nucleic acid sequence.

### vi. Identifying Barcode Equals Identifying Candidate Compound That Regulates Target RNA

In some aspects, the identification, based on sequencing, of the barcode in each cell can identify (e.g. corresponds to) the candidate compound contacted with that cell. Thus, in some aspects, there is a direct correlation between the barcode and candidate compound. In some aspects, the barcode is specific to a candidate compound and the candidate compound is responsible for regulating a regulatory factor that can affect the target RNA.

In some aspects, the regulatory factor can be any molecule that can alter the abundance of a target RNA. In some aspects, the regulatory factor can be, but is not limited to, a transcription factor, kinase, G protein, splicing machinery, chromatin modifier, phosphatase, enzyme, metabolic gene, metabolite transporter, chaperone, scaffolding protein, ligase, signaling machinery, degradation machinery.

### vii. Target RNA can be an Example of a Target Molecule

In some aspects, the disclosed method can be used in a broader sense, wherein the method can identify a candidate compound that alters a target molecule. In some aspects, a target molecule can be, but is not limited to nucleic acids (DNA or RNA) or protein.

Disclosed method of identifying a candidate compound that regulates abundance of a target molecule comprising introducing a barcode to a population of cells; contacting the population of cells with a candidate compound; labeling the target molecule in the population of cells; identifying cells in the population of cells that have altered abundance of the target molecule; sequencing DNA from the cells having an abundance of the target molecule using primers specific to the barcode; identifying, based on sequencing, the barcode in each cell, wherein the barcode corresponds to the candidate compound contacted to the population of cells having that barcode; thereby identifying a candidate compound that regulates the abundance of the target molecule.

In some aspects, depending on the target molecule, the method of labeling can be different. For example, if the target molecule is nucleic acid based then methods such as FISH can be used for labeling. If the target molecule is protein based then labeling requires either a known nucleic acid that binds the protein or another protein, such as an antibody, that binds the target molecule.

In some aspects, all of the steps that are not specific to the target molecule can be performed as described throughout. For example, the sequencing is still performed using primers that amplify the barcode and the barcode corresponds to the candidate compound.

### C. Kits

The compositions and materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits for performing the disclosed methods, the kit comprising sgRNAs, barcodes, or primers for the disclosed sgRNAs or barcodes. The kits also can contain candidate compounds.

### EXAMPLES

### A. Example 1

### 1. TRoUT-FISH Enables Discovery of Gene Regulation

A major goal in understanding gene regulation is identifying the set of factors that control each gene's expression. Many aspects of a gene's regulation can only be understood in its native genomic context; however, this places many limitations on the types of experiments that can be performed as well as how easily they can be applied to the study of any and all genes. To address this problem, Targeted Readout to Understand Transcription via Fluorescent In Situ Hybridization (TRoUT-FISH) has been developed as a straightforward and unbiased means to identify all factors that regulate a gene at the level of transcript abundance. TRoUT-FISH—which combines fluorescent in situ hybridization, flow cytometry, and CRISPR screening—can be used as a platform to answer fundamental questions about gene regulation including disentangling redundant networks like the integrated stress response, illuminating cooperativity between transcriptional co-regulators, and annotating the regulome.

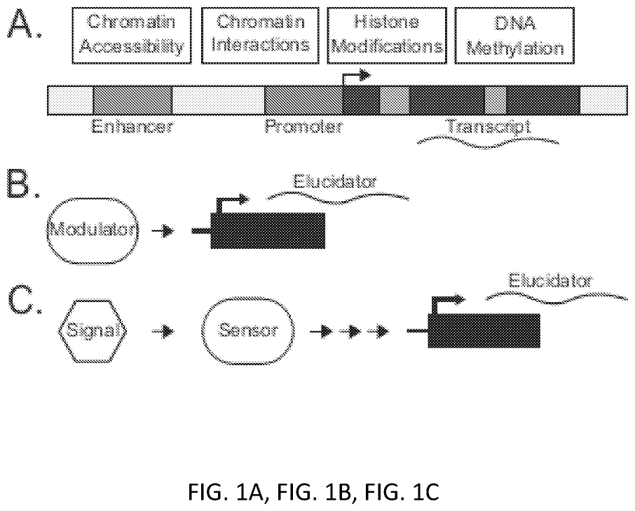

A major technological gap in understanding gene regulation is the inability to easily identify upstream regulatory factors (FIG. 1A-C). To address this issue, TRoUT-FISH, which combines CRISPR screening, FISH, and flow cytometry (FIG. 2) was developed to identify genes required for native gene regulation, including histone modifications, chromatin accessibility, and three-dimensional genome organization (FIG. 1A). Thus, factors responsible for gene regulation can be discovered without the need for exogenous reporters or genome modification—both of which are time consuming and can lead to artifactual results. This approach is capable of detecting not only trans-acting factors—such as transcription factors or chromatin modifiers—but machinery upstream in a regulatory axis including kinases and downstream of transcription including splicing factors, polyadenylation machinery, and RNA maturation factors.

One major obstacle in understanding gene regulation is the discovery of upstream regulatory factors (FIG. 1A). In order to directly define gene regulation, TRoUT-FISH was developed which combines modified fluorescent in situ hybridization (FISH) with CRISPR screening and flow cytometry. TRoUT-FISH (FIG. 2) measures an RNA of interest—the elucidator—by flow cytometry and uses pooled CRISPR screening to identify regulatory factors—the modulators—which control elucidator levels (FIG. 1B-C).

TRoUT-FISH is performed on bulk populations which sidesteps the cost and data sparsity issues common to single cell RNA-seq assays. As TRoUT-FISH experiments are performed without any genetic manipulations prior to CRISPR perturbations, the system is ideally positioned to integrate with other data, including ChIP-seq, ATAC-seq, and ENCODE resources. These other resources provide complementary mechanistic insight of gene regulation. Cells are infected at low MOI with virus encoding a CRISPR library selected based on antibiotic resistance. Cells are cultured for eight days to allow CRISPR editing to occur. Paraformaldehyde-fixed cells are permeabilized and stained with a DNA dye and incubated overnight in hybridization buffer in the presence of specific DNA oligonucleotides that anneal to an RNA of interest. The DNA oligonucleotides are computationally designed genome-wide to ensure specificity for the elucidator. Fixed cells are then incubated with fluorophore-conjugated secondary oligos and cells with lowest and highest signal are sorted by flow cytometry. For these populations, DNA is extracted, sgRNA sequence amplified by PCR, and the pooled amplicons are sequenced (FIG. 2).

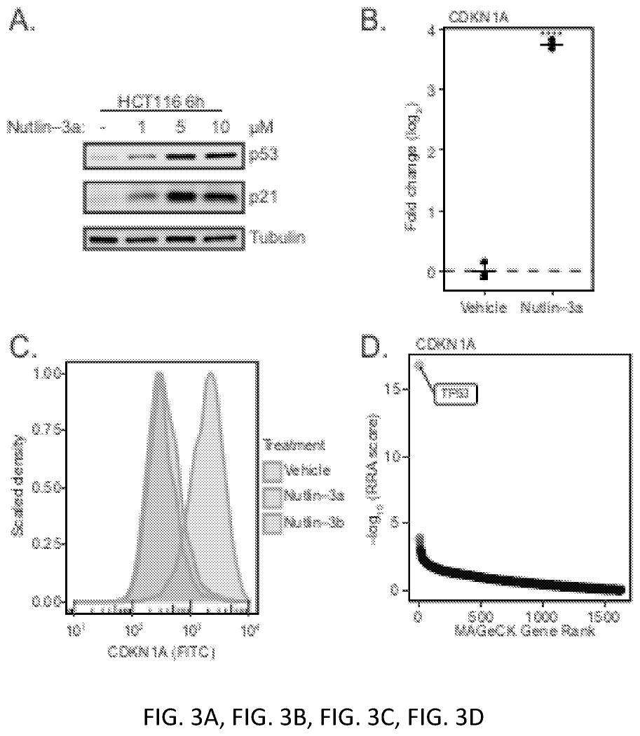

The small molecule Nutlin-3a, which inhibits the interaction between MDM2 and p53 and, in doing so, prevents the proteolytic degradation of p53 and permits activation of downstream targets such as CDKN1A, has been used. Treatment of HCT116 cells (wildtype for TP53) with Nutlin-3a resulted in both the stabilization of p53 protein and induction of CDKN1A transcript (FIG. 3 A-B). These changes were then assayed using FISH coupled with flow cytometry (FIG. 2). Cells treated with the active enantiomer (Nutlin-3a) displayed higher fluorescence than either the control or inactive enantiomer (FIG. 3C), indicating that this method can robustly detect increases in the expression of a given gene. A CRISPR screen was performed in HCT116 cells stably expressing Cas9 with a small library targeting ˜1500 transcription factors, including ten sgRNAs against TP53. After acute selection, cells were treated with vehicle or Nutlin-3a and the transcript abundance of CDKN1A was determined by FISH. CDKN1Alow and CDKN1A high populations were sorted by flow cytometry (e.g. FACS), isolated genomic DNA, and sequenced sgRNAs (FIG. 2). This screen positively identified TP53 as the critical mediator of Nutlin-3a induced CDKN1A expression (FIG. 1D). As validation, stable TP53 knockout lines were generated and no induction of CDKN1A signal upon treatment with Nutlin-3a was observed. Together, these data indicate that CRISPR screens can be performed using FISH as a proxy for RNA transcript abundance to elucidate factors responsible for gene regulation.

### 2. Transcriptional Control of Glycolytic Genes

While glycolysis is a central metabolic pathway for cellular energy generation, the understanding of it's control is incomplete. Studies have identified some transcriptional regulators of glycolytic genes—namely MYC and HIF1A—but there has not been an unbiased and systematic investigation.

Having established the feasibility of using TRoUT-FISH to detect gene regulatory factors, the control of glycolysis was interrogated. A cellular system was sought in which all glycolytic genes were expressed at appreciable levels. To this end, DepMap gene expression data was queried for >1000 cell lines. Expression levels were extracted for glycolytic genes and scored based on cumulative expression. Interestingly, the top one hundred scoring cell lines were enriched for cells from either skin (P-value=1.832e-4, Fisher's exact test) or central nervous system (P-value=2.1e-06, Fisher's exact test) lineages. Two distinct cell systems were selected: a skin cell line (MeWo) which express each gene of glycolysis at high levels (data not shown), and a chronic myelogenous leukemia cell line (K562) which has been extensively used for screening and genomics assays.

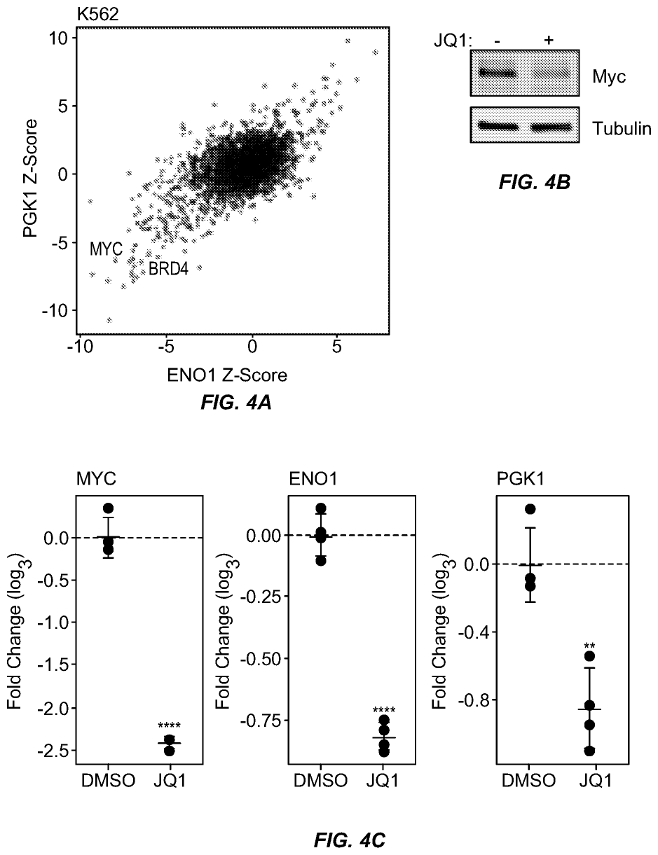

First, TRoUT-FISH experiments were performed assaying the regulation of ENO1 and PGK1 in K562 cells (FIG. 4A). Analysis of the screen data revealed that MYC and BRD4 were common modulators. MYC is a well-characterized activator of glycolytic gene expression, and as such served as an internal positive control. BRD4 is a member of the bromodomain and extraterminal domain (BET) family of proteins and is known to regulate MYC at the transcript level. To validate this screen hit, K562 cells were treated with JQ1 (a well-studied inhibitor of BRD4) and measured MYC protein level (FIG. 4B) as well as MYC, ENO1, and PGK1 transcript levels (FIG. 4C) which were all significantly downregulated. Taken together, these data indicate the screen accurately captured known regulatory events.

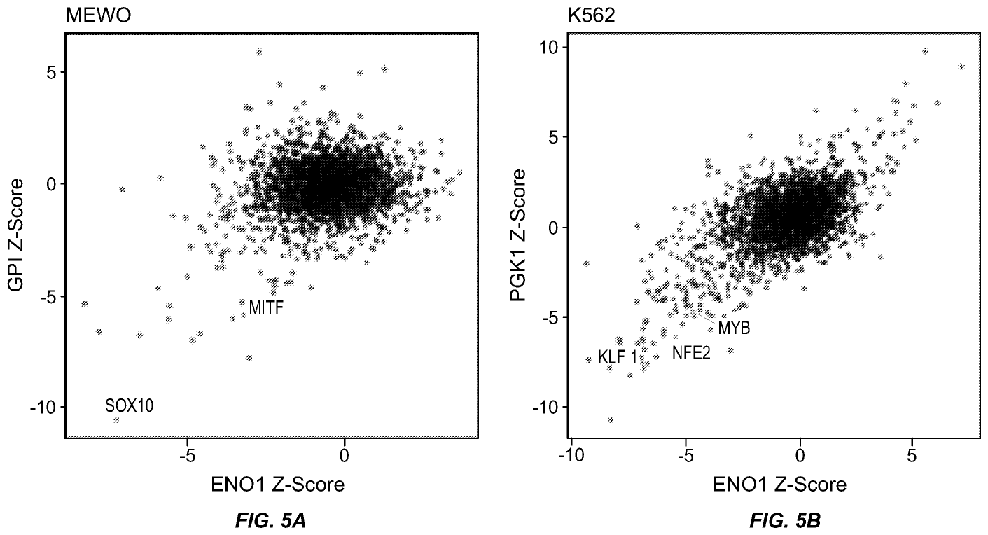

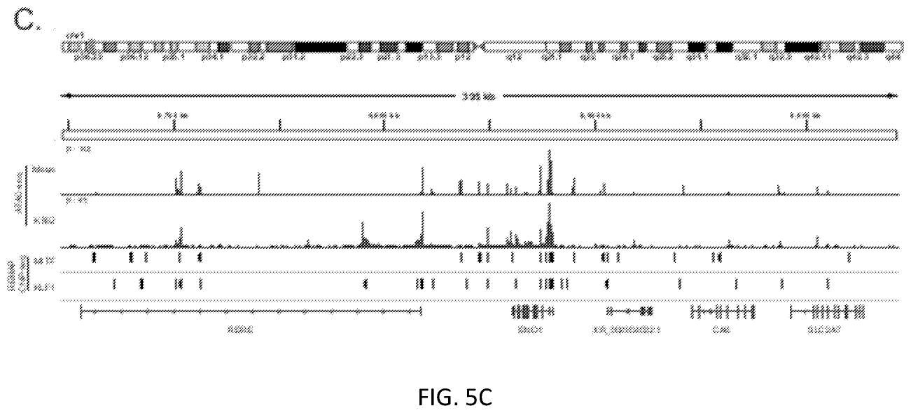

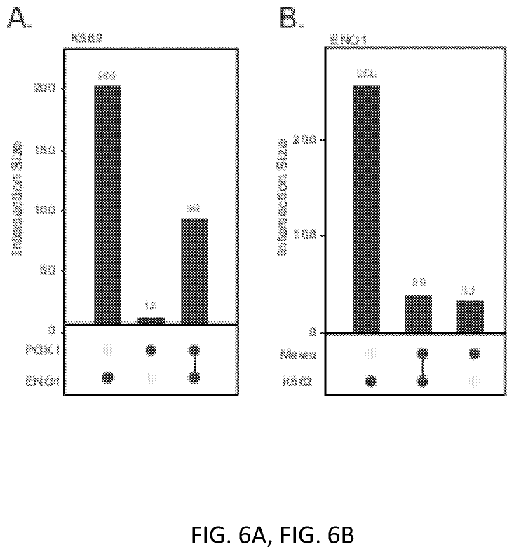

Next, TRoUT-FISH experiments were performed to determine what factors might control expression of ENO1 and GPI in MeWo cells. Interestingly, when comparing these screens, lineage defining transcription factors (TFs) were identified as statistically significant regulators (FIG. 5A-B). These included SOX10 and MITF in the skin lineage and KLF1, NFE2, and MYB in the blood lineage. These experiments can determine if lineage specific TFs are responsible for control of glucose metabolism. First, TRoUT-FISH experiments can be carried out in these two cell lines for additional glycolytic genes (HK1, GPI, PFKM/P, ALDOA, PGK1, ENO1, and PKM). These data can provide a unique understanding of how glycolytic genes are controlled and inform about coordinated control across the entire pathway. Indeed, data has already revealed many shared regulators for ENO1 and PGK1 in the same cell system (FIG. 6A), and even a shared set of regulators for ENO1 across cell lines (FIG. 6B). Critically these, data indicate that a core regulatory paradigm might exist that coordinates many glycolytic genes, with flexibility gained from lineage specific TFs. ATAC-seq and ChIP-seq data indicate putative cis-regulatory regions in both cell models (FIG. 5C).

### 3. Regulation of Nutrient Responsive Genes

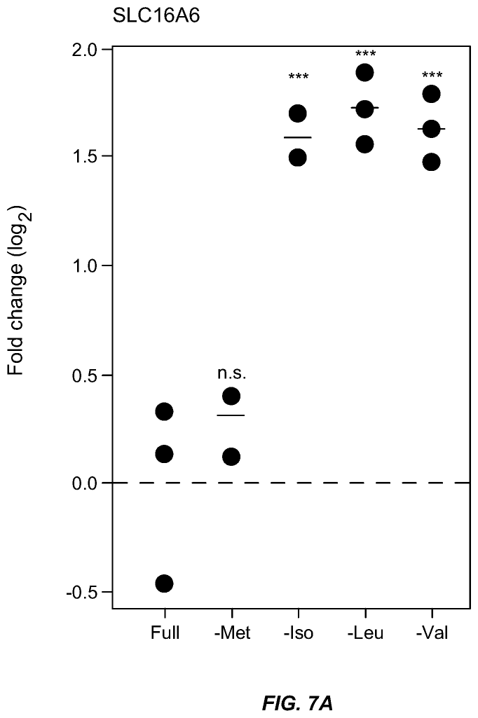

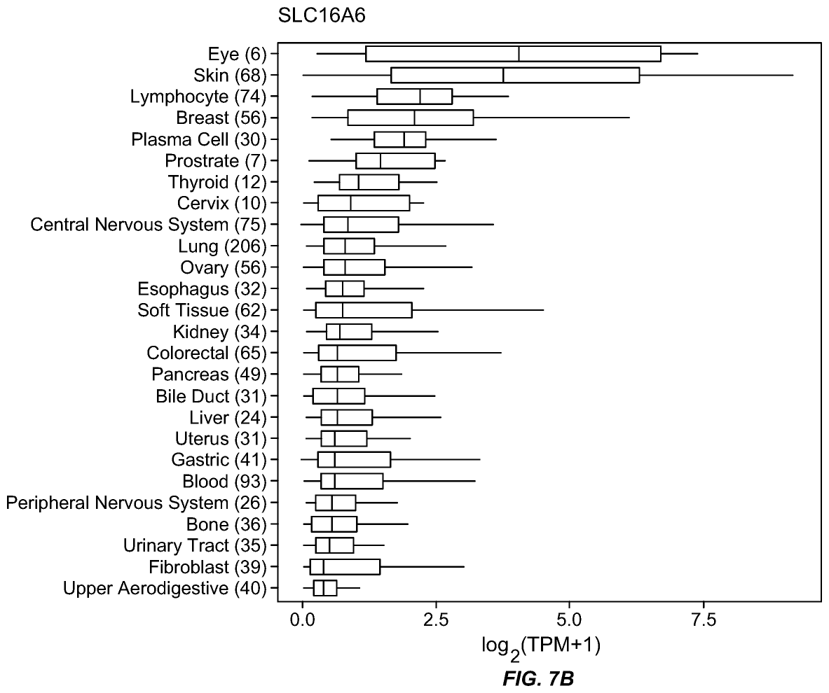

In response to diverse stresses, such as limitations in nutrients, cells reprogram the transcriptome to enact changes in cellular behavior. To identify amino acid responsive genes, cells were cultured in medium with physiological levels of nutrients before acutely removing single proteinogenic amino acids and performing RNA-seq. Expression of SLC16A6 was upregulated in response to deprivation of valine, leucine, and isoleucine but not methionine or other amino acids (FIG. 7A). SLC16A6 is uncharacterized in mammals but, based on similarity, it is predicted to be a proton-linked monocarboxylate transporter. SLC16A6 can be induced specifically in response to branched chain amino acid (BCAA) limitation.

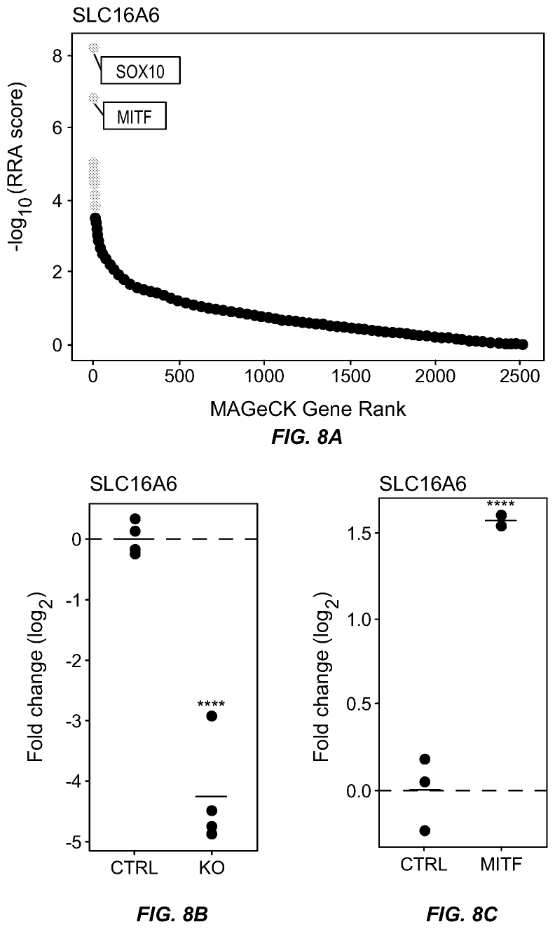

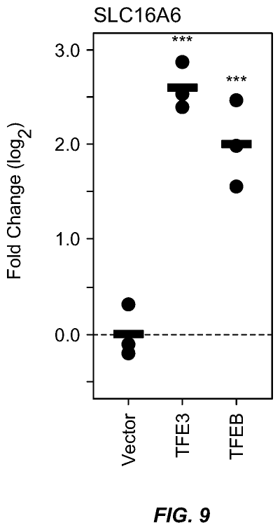

SLC16A6 is uncharacterized in mammals but is expressed in a lineage-selective manner, predominately in skin cells (FIG. 7B) and, as described above, in response to BCAA deprivation (FIG. 7A). TRoUT-FISH can be used to determine modulators in both scenarios. Data from a SLC16A6 TRoUT-FISH experiment (using an epigenetic focused library) indicates that SOX10 and MITF act as potential modulators (FIG. 8A). MITF is a member of the nutrient-responsive MiT/TFE family of basic helix-loop-helix leucine zipper transcription factors. This family includes TFEB, TFE3, and TFEC. Interestingly MITF displays significant genetic complexity with several splice variants and protein isoforms. To validate MITF modulation of SLC16A6, published RNA-seq data was reprocessed from MITF knockout cells (FIG. 8B) and MITF overexpression (FIG. 8C), which revealed a significant decrease or increase in SLC16A6, respectively. Additionally, exogenous expression of TFE family members (FIG. 9) can increase expression of SLC16A6. Taken together these data indicate that TFE/MiT family members are necessary and sufficient to regulate SLC16A6.

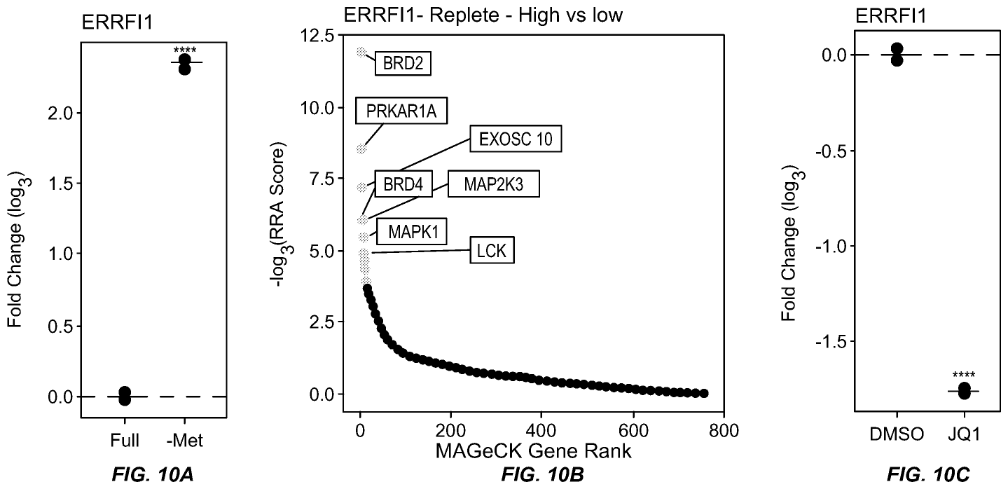

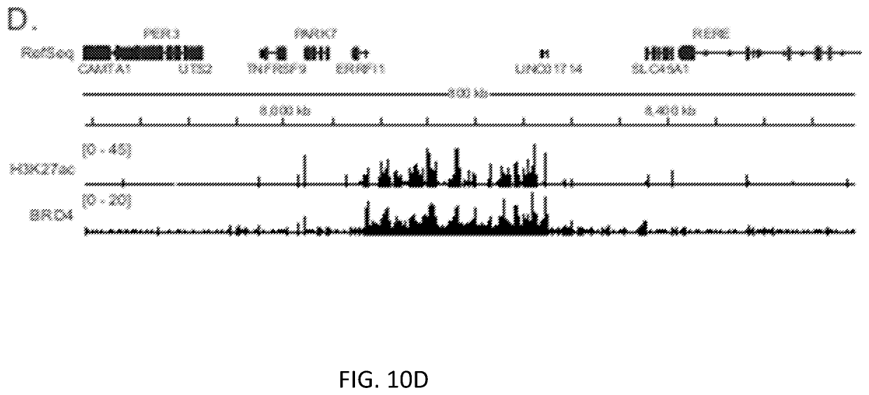

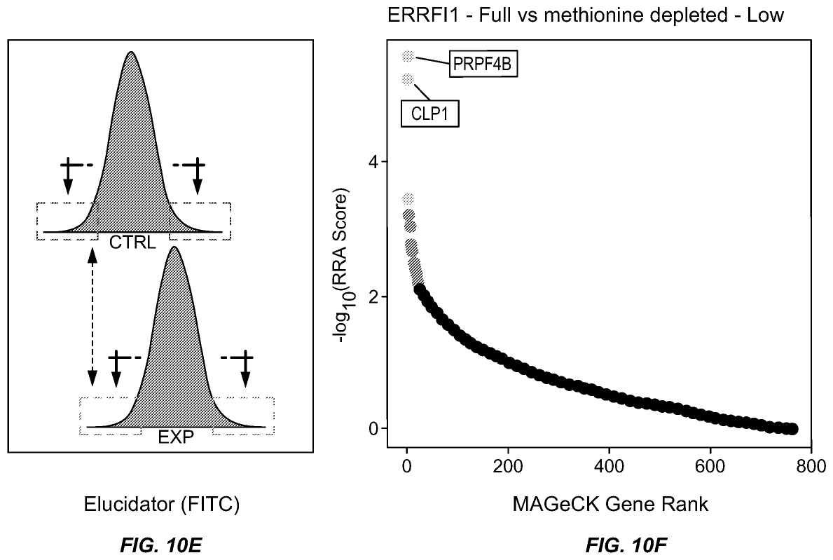

The RNA-seq data also indicate that ERRFI1 is transcriptionally upregulated upon amino acid deprivation (FIG. 10A). ERRFI1 has been described in the literature as a negative regulator of Epidermal Growth Factor Receptor (EGFR) signaling although some data indicates the role of ERRFI1 may be tissue dependent. A kinome specific TRoUT-FISH experiment in HCT116 cells was identified to identify modulators of ERRFI1 which identified the BET family of proteins—BRD2 and BRD4 (FIG. 10B). Based on the within group analysis, these modulators are expected to control ERRFI1 in nutrient rich settings (FIG. 10E, magenta). BRD family members are transcriptional and epigenetic regulators which contain two bromodomains which bind acetylated lysines on proteins, including histones. To test for changes in ERRFI1 levels upon BRD inhibition RNA-seq data was reprocessed from HCT116 cells treated with JQ1, which revealed a reduction in ERRFI1 levels (FIG. 10C). Next, public ChIP-seq data was mined for HCT116 cells profiled for both H3K27ac and BRD4. The genome browser shot displays highly correlative peaks for both tracks (H3K27ac and BRD4), in a pattern which is typically indicative of a super-enhancer (FIG. 10D).

In parallel with the replete TRoUT-FISH experiment, an acute (4 hour) methionine deprivation experiment was completed. The sgRNA abundance in the low tail of the replete sample was compared to the low tail of the nutrient-depleted sample (FIG. 10E magenta vs cyan). Comparisons between groups (different color dashed boxes in FIG. 10E) reveal modulators involved in stimulus mediated transcription. With this analysis, two modulators were identified. PRPF4B has been implicated in several cellular activities ranging from pre-mRNA splicing to control of Hippo signaling in the nucleus via phosphorylation of YAP/TAZ. CLP1 has similarly been described to function in several biological processes including pre-mRNA cleavage and tRNA maturation. Interestingly, GCN2 was not identified in the analysis, indicating GCN2-independent transcriptional regulation of ERRFI1. Together these data indicate that a nutrient stress response exists that is triggered by the removal of several distinct amino acids of which ERRFI1 is a target. This program is independent of two major nutrient sensing pathways controlled by GCN2 and TOR. In a single experiment, TRoUT-FISH was able to rule out regulation by GCN2 and TOR, and point to a novel mechanism of regulation by CLP1 and PRPF4B.

### B. Example 2

### 1. MACAREL-FISH Facilitates Phenotypic Screening of RNA Abundance

High-throughput screening is an essential tool in determining small molecule compounds that produce a desired effect in cells towards a therapeutic avenue. Crude phenotypes such as cell viability or growth are routine, however, large scale phenotypic screening are limited by the type of phenotype one can easily and accurately measure. In particular, measurements of gene expression are difficult to scale. To solve this problem, a Method to Associate Compounds with Altered RNA levels via Fluorescent In Situ Hybridization (MACAREL-FISH) was developed. MACAREL-FISH allows high-throughput screening to identify small molecules (drugs, metabolites, compounds, fragments) with changes in RNA levels. This allows an investigator to test many thousands of compounds simultaneously for the ability to alter (increase or decrease) a given RNA.

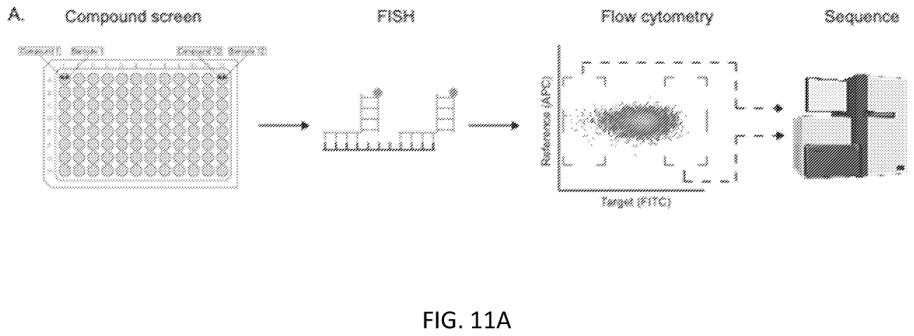

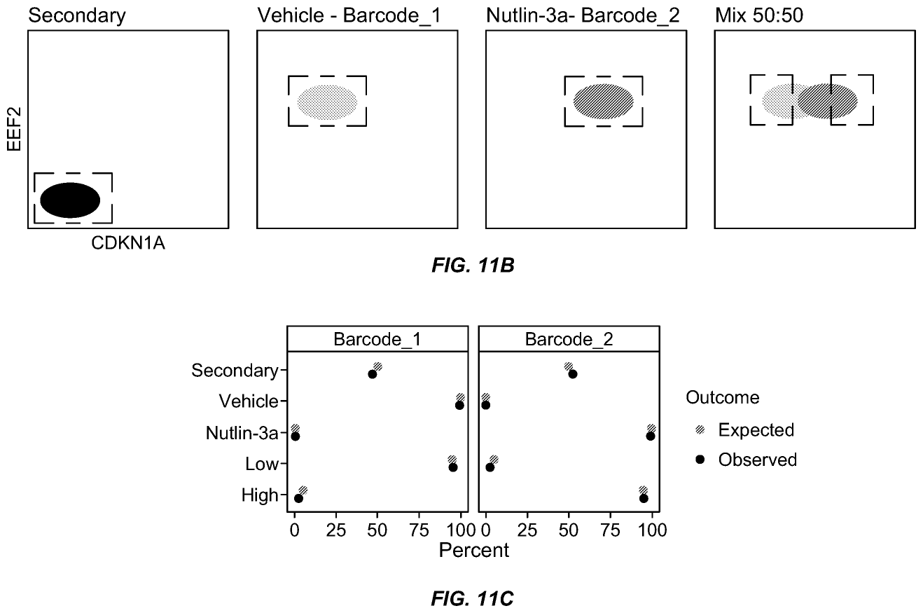

In brief, cells are arrayed in a 96 well plate (FIG. 11A). MACAREL-FISH is amenable to any cell type including: immortalized cancer cell lines, primary cells, patient cells, and organoids. Each well is incubated with a specific compound. Before, during, or after treatment cells are barcoded (FIG. 11A). Cells are then detached from the plate and fixed with paraformaldehyde (PFA) which locks cells in their current state. Cells are then pooled permeabilized and stained with a DNA dye and incubated overnight in hybridization buffer in the presence of specific DNA oligonucleotides that anneal to an RNA of interest. The DNA oligonucleotides are computationally designed genome-wide to ensure specificity for the elucidator. Fixed cells are then incubated with fluorophore-conjugated secondary oligos and cells are sorted into tubes according to the expression level of their target gene. For these populations, DNA is extracted, barcode sequence amplified by PCR, and the pooled amplicons are sequenced. In this way we can associate a unique barcode (which is representative of drug treatment) with the expression of the target RNA. Based on the possible sequence space of barcodes and read length on NGS, this approach can be scaled almost ad infinitum.

Cells were treated with either vehicle control or the small molecule Nutlin-3a. These two populations of cells received unique barcodes (FIG. 11B). After treatment, cells were fixed and FISH was performed on either vehicle cells, treated cells, or an equal mixture of both populations (FIG. 11B). These samples were processed by FACS to sort the indicated populations (FIG. 11B). After sorting, DNA was extracted and barcodes identity and abundance were measured by sequencing. The data shows that measured data matches with expected barcode identity and abundance, indicating that MACAREL-FISH is an effective method to link small molecules with molecular changes, particularly changes in RNA abundance (FIG. 11C).

Those skilled in the art will recognize, or be able to ascertain using no more than routine experimentation, many equivalents to the specific embodiments of the method and compositions described herein. Such equivalents are intended to be encompassed by the following claims.

We claim:

1. A method of identifying a regulatory factor that regulates the abundance of a target RNA comprising:
a. introducing to a population of cells a plurality of single guide RNAs (sgRNAs), wherein the sgRNAs are specific for one or more nucleic acid sequences capable of encoding the regulatory factor in the population of cells;
b. performing fluorescent in situ hybridization (FISH) on the population of cells, using a probe specific to a target RNA in the population of cells;
c. identifying cells in the population of cells that have altered abundance of the target RNA;
d. sequencing DNA from the population of cells that have altered abundance of the target RNA; and
e. identifying, based on the sequencing, sgRNAs that are specific to a nucleic acid sequence capable of encoding the regulatory factor that regulates abundance of the target RNA;
thereby identifying a regulatory factor that regulates abundance of the target RNA.

2. The method of claim 1, wherein the probe is fluorescently labeled.

3. The method of any of the preceding claims, further comprising, before step c), contacting the population of cells with a labeled probe specific to the probe.

4. The method of claim 1, wherein the regulatory factor is a transcription factor, a kinase, a G protein, splicing machinery, a chromatin modifier, a phosphatase, an enzyme, a metabolic gene, a chaperone, a scaffold, ligase, or degradation machinery.

5. The method of any of the preceding claims, wherein the cells comprise a Cas protein.

6. The method of any of the preceding claims, further comprising contacting the population of cells with a Cas protein or a gene capable of expressing a Cas protein prior to performing FISH.

7. The method of claim 5 or 6, wherein the Cas protein is Cas9.

8. The method of any of the preceding claims, wherein introducing a plurality of sgRNAs comprises introducing, to the second population of cells, a virus library, wherein each virus comprises a sgRNA.

9. The method of claim 8, wherein sequencing comprises contacting the DNA from the population of cells that have altered abundance of the target RNA with primers specific to a constant region upstream and downstream of the sgRNA on the plasmid.

10. The method of any of the preceding claims, wherein the plurality of sgRNAs are from a library with known targets.

11. The method of any of the preceding claims, wherein identifying cells in the population of cells that have altered abundance of the target RNA comprises performing fluorescence activated cell sorting (FACS).

12. The method of any of the preceding claims, wherein the population of cells are human cells.

13. The method of any of the preceding claims, wherein the population of cells are cancer cells.

14. The method of any of the preceding claims, wherein the population of cells are healthy cells.

15. A method of identifying a regulatory factor that regulates abundance of a target RNA effected by a candidate compound comprising:
a. contacting a first population of cells with a candidate compound;
b. identifying a target RNA that is modulated by the candidate compound;
c. preparing a probe specific to the target RNA identified in step b);
d. introducing to a second population of cells a plurality of single guide RNAs (sgRNAs), wherein the sgRNAs are specific for one or nucleic acid sequences capable of encoding the regulatory factor in the second population of cells;
e. performing fluorescent in situ hybridization (FISH) on the second population of cells, using the probe of step c);
f. identifying cells in the second population of cells that have altered abundance of the target RNA;
g. sequencing DNA from the cells of step having altered abundance of the target RNA;
h. identifying, based on the sequencing, sgRNAs that are specific to a nucleic acid sequence capable of encoding the regulatory factor that regulates the abundance of the target RNA, and
thereby identifying a regulatory factor that regulates abundance of the target RNA effected by the candidate compound.

16. The method of claim 15, wherein the regulatory factor is a transcription factor, a kinase, a G protein, splicing machinery, a chromatin modifier, a phosphatase, an enzyme, a metabolic gene, a chaperone, a scaffold, ligase, or degradation machinery.

17. The method of any one of claims 15-16, wherein identifying a target RNA that is modulated by the candidate compound comprises performing RNA sequencing.

18. The method of any one of claims 14-17, wherein the probe is fluorescently labeled.

19. The method of any one of claims 14-17, further comprising, before step c), contacting the population of cells with a labeled probe specific to the probe.

20. The method of any of the preceding claims, wherein introducing a plurality of sgRNAs comprises introducing, to the second population of cells, a virus library, wherein each virus comprises a sgRNA.

21. The method of claim 20, wherein sequencing comprises contacting the DNA from the population of cells that have altered abundance of the target RNA with primers specific to a constant region upstream and downstream of the sgRNA on the plasmid.

22. The method of any one of claims 15-21, wherein identifying cells in the second population of cells that have altered abundance of the target RNA comprises performing fluorescence activated cell sorting (FACS).

23. The method of any of the preceding claims, wherein the plurality of sgRNAs are from a library with known targets.

24. The method of any of the preceding claims, wherein the cells of the second cell population comprise a Cas protein.

25. The method of any of the preceding claims, further comprising contacting the population of cells with a Cas protein or a gene capable of expressing a Cas protein prior to performing FISH.

26. The method of claims 24 or 25, wherein the Cas protein is Cas9, Cas12, Cas13 or dCas9.

27. The method of any of the preceding claims, wherein the population of cells are mammalian cells.

28. The method of any of the preceding claims, wherein the population of cells are cancer cells or cells having a metabolic disease/condition.

29. The method of any of the preceding claims, wherein the population of cells are healthy cells.

30. The method of any of the preceding claims, wherein the first population of cells and second population of cells are the same cell type.

31. The method of any of the preceding claims, wherein the candidate compound is a known or unknown compound.

32. A method of identifying a candidate compound that regulates abundance of a target RNA comprising
a. introducing a barcode to a population of cells;
b. contacting the population of cells with a candidate compound;
c. performing fluorescent in situ hybridization (FISH) on the population of cells of step b), using a probe specific to a target RNA in the population of cells;
d. identifying cells in the population of cells that have altered abundance of the target RNA;
e. sequencing DNA from the cells of step d) using primers specific to the barcode;
f. identifying, based on sequencing, the barcode in each cell, wherein the barcode corresponds to the candidate compound contacted to the population of cells having that barcode;
thereby identifying a candidate compound that regulates the abundance of the target RNA.

33. The method of claim 32, wherein step a) is performed prior to step b).

34. The method of claim 32, wherein step b) is performed prior to step a).

35. The method of claim 32, wherein step a) and step b) are performed simultaneously.

36. The method of any one of claims 32-35, wherein introducing the barcode comprises administering a plurality of DNA constructs to the population of cells, wherein each DNA construct comprises a unique barcode.

37. The method of claim 36, wherein the plurality of DNA constructs is a plurality of plasmids.

38. The method of any one of claims 36-37, wherein sequencing comprises contacting the DNA from the population of cells that have altered abundance of the target RNA with primers specific to a constant region upstream and downstream of the barcode on the DNA construct.

39. The method of any one of claims 32-38, wherein the probe is fluorescently labeled.

40. The method of any one of claims 32-39, further comprising, before step d), contacting the population of cells with a labeled probe specific to the probe.

41. The method of any one of claims 32-40, wherein identifying cells in the population of cells that have altered abundance of the target RNA comprises performing fluorescence activated cell sorting (FACS).

42. The method of any one of claims 32-41, wherein the regulatory factor is a transcription factor, a kinase, a G protein, splicing machinery, a chromatin modifier, a phosphatase, an enzyme, a metabolic gene, a chaperone, a scaffold, ligase, or degradation machinery.

43. The method of any one of claims 32-42, wherein the population of cells are human cells.

44. The method of any one of claims 32-43, wherein the population of cells are cancer cells.

45. The method of any one of claims 32-43, wherein the population of cells are healthy cells.
